(19) United States
(12) Patent Application Publication
(10) Pub. No.: US 2022/0193025 A1
(43) Pub. Date: Jun. 23, 2022

(54) USE OF DIANHYDROHEXITOL TO ELIMINATE THE COSMETIC EFFECTS OF ACNE, DANDRUFF AND BAD ODORS

(71) Applicant: ROQUETTE FRERES, Lestrem (FR)

(72) Inventors: Léon MENTINK, Lille (FR); Daniel WILS, Morbecque (FR)

(21) Appl. No.: 17/593,727

(22) PCT Filed: Mar. 27, 2020
(86) PCT No.: PCT/EP2020/058678

§ 371 Date: Sep. 23, 2021

(30) Foreign Application Priority Data
Mar. 28, 2019 (FR) 19 03286
Mar. 28, 2019 (FR) 19 03288
Mar. 28, 2019 (FR) 19 03289

(51) Int. Cl. A61K 31/34; A61P 17/10; A61Q 5/00; A61Q 17/00; A61K 8/49

(52) U.S. Cl. CPC A61K 31/34; A61P 17/10; A61K 8/4973; A61Q 17/005; A61Q 5/006

(57) ABSTRACT

The present application pertains to the field of cosmetics, more specifically the field of antimicrobial and/or bacteriostatic and/or bactericidal cosmetic agents, preferentially acting on bacterial and/or fungal strains on the human epidermis. Proposed is a use of dianhydrohexitol, preferentially isosorbide, to reduce the number of bacterial or fungal strains on the skin, in particular in the cutaneous microbiome, causing acne, dandruff buildup and bad odors.

### TECHNICAL FIELD

The present application pertains to the field of cosmetics, more specifically the field of antimicrobial and/or bacteriostatic and/or bactericidal and/or antifungal cosmetic agents, preferentially acting on bacterial and/or fungal strains on the human epidermis. Preferentially, the application proposes a use of isosorbide to reduce the number of bacterial or fungal strains present on the skin, in particular in the cutaneous microbiome, causing acne, dandruff buildup and bad odors; or to increase the activity of biocides already used for this purpose.

### BACKGROUND ART

In U.S. Pat. No. 9,295,626 by Pilz et al., a method for preserving cosmetic, dermatological and pharmaceutical preparations by the use of bactericidal compounds based on isosorbide monoester and/or diester is disclosed. This patent has a minimum inhibitory concentration for the isosorbide equal to 10% on many strains (column 27, table 2), including in particular the following strains that can be found in the cutaneous microbiome: Aspergillus brasiliensis, Candida albicans, Staphylococcus aureus.

U.S. Pat. No. 999,300 by Stoer et al. likewise discloses the use of isosorbide derivatives, in this case ethers, as bactericidal agent for preserving cosmetic compositions, in particular by inhibiting the growth of multiple bacterial strains, including Propionobacterium acnes.

In the pharmaceutical preparations for treating acne in U.S. Pat. No. 6,433,024 by Popp et al., isosorbide or its alkyl derivatives, are mentioned as moisturizing agent to reduce the irritation induced by the anti-acne active agent, which is benzoyl peroxide. Only dimethyl isosorbide is given as an example in topical formulations, and only as a moisturizing agent, since benzoyl peroxide is the active agent always used.

The solution provided by the present invention makes it possible to solve the problems raised by a known solution which is salicylic acid or benzoyl peroxide, which have the major disadvantages of causing irritation of the epidermis, or which has a certain toxicity that was recently discovered.

### Technical Problem

Human skin is colonized by a resident bacterial flora, which constitutes the cutaneous microbiome. Although this flora is non-pathogenic most of the time, in certain abnormal situations it may become pathogenic. A first abnormal situation is that of contamination of the cutaneous microbiome by a pathogenic external microorganism, through contact with a contaminated environment. A second abnormal situation is that of the appearance of an imbalance in the interactions between the microorganisms that constitute the cutaneous microbiome, which results in the proliferation of one microorganism to the detriment of the other microorganisms. Such a proliferation can be generalized over a large area of the skin, or can be more localized, for example in the areas of the skin that are richer in water or warmer, or for example on the face or scalp due to the presence of nutritional reserves for said microorganisms, such as the triglycerides located in the glands at the base of the hair or hairs. Apart from the potentially harmful consequences for the health of the skin or of the individual, these abnormal situations also cause disorders of a cosmetic nature, in particular visual, tactile, or olfactory, which can affect the individual's comfort or their image, and thus disrupt or degrade their social life.

The bacterial microorganism Propionobacterium acnes, currently also referred to as Cutibacterium acnes, causes acne. Acne is a skin disorder characterized by excessive sebum secretion by the sebaceous glands. The sebum reaches the surface of the skin through the hair follicle duct. The excessive presence of sebum blocks the passage of the hair follicle duct. This causes the sebum to thicken, thus forming a comedo (blackhead or whitehead). This comedo is contaminated by the cutaneous microbiome, in particular the Propionobacterium acnes bacterium. The latter grows and multiplies in the sebum that has built up in the comedo. Sebum is actually a source of nutrition for this bacterium, which then releases metabolites into the skin pore. These chemical products alert and attract white blood cells, leading to inflammation, visible by redness for example at the comedones, and felt as itchy skin, specifically localized on the acne comedones.

Inflammation can damage the collagen-producing cells. A drop in collagen production can lead to thinning of the skin, causing sunken scars (also referred to as depressed scars). Sometimes, inflammation leads to increased collagen production, which causes a thickening of the scars. The present invention makes it possible to improve the structure of the scars while avoiding redness and itching.

The fungal microorganisms of the Malassezia spp family, which include Malassezia sympodialis, Malassezia obtusa, Malassezia slooffiae, Malassezia restricta, Malassezia globosa and Malassezia furfur, cause dandruff buildup (Frederick Manuel, S Ranganathan. A new postulate on two stages of dandruff: a clinical perspective. Int J Trichology. 2001; 3(1):3-6, Shivaprakash M Rudramurthy, Prasanna Honnavar, Sunil Dogra, Prakash P Yegneswaran, Sanjeev Handa, Arunaloke Chakrabarti. Association of Malassezia species with dandruff. Indian J med Res. 2014,139(3):431-437). The Malassezia are microscopic yeasts naturally present on the skin surface. These yeasts are particularly abundant in the areas of the body that are rich in sebaceous glands such as the scalp. Half of all people affected have no consequences from malassezia, while the other half suffers from excessive dandruff buildup.

Indeed, for example among people with greasy hair, Malassezia spp proliferate on the scalp and feed off the sebum, producing fatty acids which are particularly irritating for the skin. This proliferation thus leads to inflammation, which prevents complete maturing of the keratinocytes, so that they prematurely detach from the scalp in the form of large clusters of cells, commonly referred to as “dandruff”.

Inflammation is generally accompanied by excessive redness and intense itching of the scalp.

Therapeutic treatments of dandruff are generally aimed at eliminating the dandruff formed, or at inhibiting the overproduction of immature keratinocytes, or even at inhibiting the growth of fungal microorganisms involved in the dandruff formation mechanism, in particular Malassezia spp. They are based for example on the use of salicylic acid, or antimycotic active ingredients having good antifungal activity against Malassezia spp, substances based on carbon, sulfur or selenium disulfide.

Some of these treatments have significant disadvantages. Salicylic acid for example causes scalp irritation. Conventional antimycotics mostly have poor renal elimination.

In addition to water and salts, sweat contains many organic substances, such as fats, amino acids, sugars, lactic acid, and urea. Fresh sweat is odorless. The typical sweat smell forms only under the action of bacteria from the cutaneous microbiome, which break the sweat down into odorous substances. For example, it is known that bacteria-type microorganisms such as Corynobacterium xerosis and Staphylococcus epidermidis, and yeasts such as Malassezia contribute to bad body odors, by breaking down body perspiration and its components, into odorous chemical compounds such as amine or sulfur compounds, often perceived as unpleasant or even nauseating odors. For these reasons, antimicrobial substances, also referred to as bactericides, are used in deodorant and antiperspirant cosmetic products, for the purpose of controlling the growth of bacteria that cause odors, often perceived as bad or nauseating odors.

Consumers nowadays are increasingly requesting products of natural origin. The use according to the invention addresses this need, proposing the use of at least dianhydrohexitol, since dianhydrohexitols are molecules of natural origin, produced from cereal-based starch, for example.

### DESCRIPTION OF EMBODIMENTS

It is to the applicant's credit to have discovered, unexpectedly, that non-derived dianhydrohexitols, preferentially isosorbide, have antimicrobial, and/or bactericidal and/or bacteriostatic, and/or antifungal effects on microbial strains that cause acne, dandruff buildup and bad body odors.

The use according to the invention makes it possible to inhibit the growth of pathogenic bacterial and/or fungal strains involved in acne, dandruff buildup and bad body odor formation, on the human epidermis, until these strains disappear from the human epidermis. The present invention relates to the use of at least one dianhydrohexitol as bacteriostatic and/or bactericidal and/or antifungal agent. Preferentially, the at least one dianhydrohexitol has:

bacteriostatic and/or bactericidal action on a bacterial strain selected among the Propionobacterium spp family, preferentially on the Propionobacterium acnes bacterial strain and/or on a strain of the Corynebacterium spp family, preferentially on the Corynebacterium xerosis bacterial strain, and/or on a strain of the Staphylococcus spp family, preferentially on the Staphylococcus epidermidis bacterial strain, and/or

action on a strain of the Malassezia spp family, preferentially on the Malassezia furfur strain.

Use of Dianhydrohexitol in the Treatment of Acne

The present invention relates to the non-therapeutic cosmetic use of at least one dianhydrohexitol to promote the elimination of the cosmetic effects of acne. The cosmetic effects of acne being selected among inflammation or itching. The invention also relates to dianhydrohexitol for use in the therapeutic treatment of acne. Preferentially the dianhydrohexitol is isosorbide.

According to another aspect, the invention proposes a use of isosorbide to reduce or eliminate the strains of Propionobacterium acnes (also referred to as Cutibacterium acnes) from the human microbiome, and also to attenuate or get rid of the cosmetic symptoms of acne, in particular redness, irritation and itching.

Preferentially, the use according to the invention makes it possible to reduce or eliminate inflammation, visible as redness for example at the acne comedones, and/or to reduce or eliminate skin itching localized on the acne comedones.

According to one preferred embodiment, the dianhydrohexitol is selected among isosorbide, isomannide, isoidide, preferentially isosorbide.

Additionally, the dianhydrohexitol concentration applied to the skin is at least 1 mg/cm², preferentially at least 25 mg/cm², and most preferentially at least 50 mg/cm².

According to one embodiment, dianhydrohexitol has antimicrobial action. More particularly, dianhydrohexitol has bacteriostatic and/or bactericidal and/or antifungal action, reducing or preventing the growth, or eliminating the presence of bacterial and/or fungal strains involved in acne, preferentially the presence of bacterial strains. Preferentially, the bacterial strain is selected among the commensal propionic bacteria, the genus of which is commonly referred to as Propionobacterium spp, preferentially the bacterial strain is Propionobacterium acnes.

According to another embodiment of the use according to the invention, dianhydrohexitol is an agent to control the unwanted effects that commensal propionic bacteria have on the skin, preferentially of the Propionobacterium spp genus, and more preferentially Propionobacterium acnes.

According to an alternative use according to the invention, the at least one dianhydrohexitol can be used in combination with at least one other cosmetic or dermatological active agent having the same activity or function.

Use of Dianhydrohexitol in the Treatment of Dandruff

The present invention relates to the non-therapeutic cosmetic use of at least one dianhydrohexitol to promote the elimination of the cosmetic effects of excessive flaking. The cosmetic effects of excessive flaking being selected among inflammation or itching. The invention also relates to dianhydrohexitol for therapeutic use thereof in the treatment of dandruff. Preferentially the dianhydrohexitol is isosorbide.

According to another aspect, the invention proposes a use of isosorbide to reduce or eliminate the strains of Malassezia furfur from the human microbiome, in order to get rid of dandruff from the scalp, or to attenuate the buildup thereof, or to make it less visible by reducing its size or amount

Preferentially, the use according to the invention makes it possible to get rid of dandruff from the scalp, or to attenuate the buildup thereof, or to make it less visible by reducing its size or amount.

According to one preferred embodiment, the dianhydrohexitol is selected among isosorbide, isomannide, isoidide, preferentially isosorbide.

Additionally, the dianhydrohexitol concentration applied to the skin is at least 1 mg/cm², preferentially at least 25 mg/cm², and most preferentially at least 50 mg/cm².

According to one embodiment, dianhydrohexitol has antimicrobial action. More particularly, dianhydrohexitol has bacteriostatic and/or bactericidal and/or antifungal action, reducing or preventing the growth, or eliminating the presence of bacterial and/or fungal strains involved in excessive skin flaking, preferentially in dandruff buildup in the scalp. Preferentially, the fungal strain is selected among the fungal strains of the Malassezia spp family, preferentially the fungal strain is Malassezia furfur.

According to another embodiment of the use according to the invention, dianhydrohexitol is an agent to control the unwanted effects that the fungal strains of the Malassezia spp family have on the skin and the scalp, preferentially on the Malassezia furfur bacterial strain. Among these unwanted effects, are dandruff buildup as mentioned above, but also pityriasis versicolor, seborrheic dermatitis, pityrosporum folliculitis.

According to an alternative use according to the invention, the at least one dianhydrohexitol can be used in combination with at least one other cosmetic or dermatological active agent having the same activity or function.

Use of Dianhydrohexitol in the Treatment of Bad Body Odors

The present invention relates to the non-therapeutic cosmetic use of at least one dianhydrohexitol in order to reduce or prevent bad body odors, preferentially the bad odors resulting from the breakdown of sweat. The invention also relates to dianhydrohexitol for the therapeutic use thereof in the treatment of bad body odors. Preferentially the dianhydrohexitol is isosorbide.

According to another aspect of the invention, a use of isosorbide is proposed to reduce or eliminate the Corynebacterium xerosis, and Staphylococcus epidermidis strains from the human microbiome, in order to attenuate or prevent the appearance of bad odors resulting from the breakdown of sweat by these microorganisms. Isosorbide is used alone, or in combination with other active agents already used for this purpose.

According to one preferred embodiment, the dianhydrohexitol is selected among isosorbide, isomannide, isoidide, preferentially isosorbide.

Additionally, the dianhydrohexitol concentration applied to the skin is at least 1 mg/cm², preferentially at least 25 mg/cm², and most preferentially at least 50 mg/cm².

According to one embodiment, dianhydrohexitol has antimicrobial action. More particularly, dianhydrohexitol has bacteriostatic and/or bactericidal and/or antifungal action, reducing or preventing the growth, or eliminating the presence of bacterial and/or fungal strains involved in the formation of bad body odors. Preferentially, the bacterial strain is selected among the bacterial strains of the Corynebacterium spp family, preferentially the bacterial strain is Corynebacterium xerosis, and/or the bacterial strain is selected among the Staphylococcus spp family, preferentially the bacterial strain is Staphylococcus epidermidis, and/or the bacterial strain is selected among the Propiobacterium spp family, preferentially the bacterial strain is Propiobacterium acnes.

According to another embodiment of the use according to the invention, dianhydrohexitol is an agent for controlling the unwanted effects on the skin of bacterial strains of the Corynebacterium spp family, preferentially Corynebacterium xerosis, and/or of the Staphylococcus spp family, preferentially is Staphylococcus epidermidis, and/or of the Propiobacterium spp family, preferentially on Propiobacterium acnes. Among these unwanted effects, are bad odors as mentioned above, but also skin infections.

According to an alternative use according to the invention, the at least one dianhydrohexitol can be used in combination with at least one other cosmetic or dermatological active agent having the same activity or function.

Dianhydrohexitol

The aqueous solution in question can contain only one dianhydrohexitol, or can also contain several. These dianhydrohexitols (1,4-3,6-dianhydrohexitols) are isosorbide (1,4-3,6-dianhydrosorbitol), isomannide (1,4-3,6-dianhydromannitol), isoidide (1,4-3,6-dianhydroiditol) and the mixtures of at least two of these products. Preferentially, the aqueous solution only contains one dianhydrohexitol which is isosorbide.

In this regard, the applicant indicates that generally, the dianhydrohexitols are synthesized in the presence of water (or water is generated during their synthesis): the recovery of said dianhydrohexitol in this reaction medium immediately provides a composition in the form of an aqueous solution of dianhydrohexitol which can be used according to the invention. Dianhydrohexitol solutions can in particular be obtained according to the methods described in above-mentioned patent applications EP1287000 and WO03/043959. The choice can be made to keep all or part of the water used during the preparation of the dianhydrohexitol or to eliminate all the water so as to obtain a product in solid form that will be put back into an aqueous solution by simply adding water, which is another possibility for preparing an aqueous solution of dianhydrohexitol that can be used according to the invention.

The applicant specifies that the term “1,4-3,6-dianhydrohexitol” does not include derivatives of 1,4-3,6-dianhydrohexitol, in particular such as 1,4-3,6-dianhydrohexitol ethers or esters.

According to one preferred embodiment, the dianhydrohexitol is selected among isosorbide, isomannide, isoidide, preferentially isosorbide.

Cosmetic or Dermatological Preparation

By “cosmetic or dermatological preparation”, the applicant means any composition intended for being placed in contact with human or animal skin.

The cosmetic or dermatological preparation according to the invention comprises as active agent for the non-therapeutic treatment of acne, at least one dianhydrohexitol, preferentially selected among isosorbide, isomannide, isoidide, preferentially isosorbide.

The cosmetic or dermatological preparation according to the invention comprises as active agent for the non-therapeutic treatment of dandruff, at least one dianhydrohexitol, preferentially selected among isosorbide, isomannide, isoidide, preferentially is isosorbide.

The cosmetic or dermatological preparation for topical use according to the invention comprises as active agent for the non-therapeutic treatment of bad odors, at least one dianhydrohexitol, preferentially selected among isosorbide, isomannide, isoidide, preferentially is isosorbide.

Additionally, the dianhydrohexitol concentration applied to the skin or scalp is at least 1 mg/cm2, preferentially at least 25 mg/cm2, and most preferentially at least 50 mg/cm2.

According to one preferred embodiment, the cosmetic or dermatological preparation according to the invention comprises 0.1% to 50% by weight of dianhydrohexitol, preferentially 0.5 to 25%, more preferentially 1% to 25%, even more preferentially 2% to 15%, and most preferentially 5% to 9%.

According to a highly preferred embodiment, the cosmetic or dermatological preparation according to the invention contains as the sole antimicrobial and/or bactericidal and/or bacteriostatic and/or antifungal agent, at least one dianhydrohexitol, preferentially selected among isosorbide, isomannide, isoidide, preferentially is isosorbide.

The cosmetic or dermatological preparation according to the invention makes it possible to reduce or eliminate redness and/or itching caused by acne, to get rid of dandruff from the scalp, or to attenuate its buildup, or to make it less visible by reducing its size or amount, to reduce or prevent bad body odors, preferentially the bad odors resulting from the breakdown of sweat.

The cosmetic preparation can be a skin product, a hair product, make-up or a hygiene product.

Among the skincare products, the cosmetic preparation according to the invention can be selected among day creams, sun creams, after-sun creams, self-tanners, masks. Among the hair care products, the cosmetic preparation according to the invention is preferentially selected among shampoos, conditioners (creams, masks, lotions), styling products (sprays, gels, waxes), coloring products. Among the make-up products, the cosmetic preparation according to the invention is preferentially chosen among foundations and eye shadows. Among the hygiene products, the cosmetic preparation according to the invention is preferentially chosen among washing gels, shower gels, cleansing or make-up removing wipes, hydroalcoholic solutions or gels, soaps, deodorants, antiperspirants, body sprays, more preferentially, among deodorants or antiperspirants, which can be in stick, gel, powder or spray form.

The cosmetic or dermatological preparation can in particular be selected among anti-acne creams or lotions.

The cosmetic or dermatological preparation can in particular be selected among anti-dandruff shampoos, anti-dandruff body and hair shower gels.

Non-Therapeutic Treatment Method

The invention proposes a non-therapeutic cosmetic method for caring for the skin or the scalp, comprising the following steps:

cleaning the skin or the scalp,

applying to the skin or scalp a cosmetic or dermatological preparation comprising at least one dianhydrohexitol, preferentially isosorbide, before, during or after the appearance of the cosmetic effects of acne or bad odors or dandruff, for a duration of at least 20 minutes, preferentially at least 2 hours, and most preferentially at least 6 hours,

removing said preparation.

The invention proposes a non-therapeutic cosmetic method for caring for skin that is susceptible to acne, which comprises the steps of:

cleaning the skin,

applying to the skin a cosmetic or dermatological preparation comprising at least one dianhydrohexitol, preferentially isosorbide, before, during or after the appearance of the cosmetic effects of acne, for a duration of at least 20 minutes, preferentially at least 2 hours, and most preferentially at least 6 hours,

removing said preparation.

The invention proposes a non-therapeutic cosmetic method for caring for the scalp, which comprises the steps of:

cleaning the scalp,

applying to the scalp a cosmetic or dermatological preparation comprising at least one dianhydrohexitol, preferentially isosorbide, before, during or after the appearance of dandruff, for a duration of at least 20 minutes, preferentially at least 2 hours, and most preferentially at least 6 hours,

removing said preparation.

The invention proposes a non-therapeutic cosmetic method for caring for skin that is susceptible to bad odors, in particular linked to the breakdown of sweat by the cutaneous microbiome, comprising the steps of:

cleaning the skin,

applying to the skin a cosmetic or dermatological preparation comprising at least one dianhydrohexitol, preferentially isosorbide, preferentially under the arms or to the groin, before, during or after the appearance of bad odors, for a duration of at least 20 minutes, preferentially at least 2 hours, and most preferentially at least 6 hours,

removing said composition.

### EXAMPLE

### Example 1: In Vitro Measurements of the Effect of Isosorbide on Microbial Strains that Cause Acne

The samples are prepared under sterile conditions and deposited in microplates (2-ml wells) with the concentrations hereunder. Two analysis controls were produced:

a positive control corresponding to 0.5% Phenonip®;

a negative control consisting of 0.85% physiological saline solution.

Samples and controls are contaminated by a Propionobacterium acnes bacterial strain with around 1.43×10⁵ to 4.15×10⁵ colony forming units per milliliter, denoted cfu/ml. After contamination, the samples are carefully mixed by suction-discharge cycles in order to ensure a uniform distribution of the microorganism. The whole is incubated at 22° C. during 28 days.

The microbial population is sampled and counted at 24 hours, 7, 14, 21 and 28 days for the Propionobacterium acnes bacterial strain. The contaminated samples are collected and then deposited in serial dilutions in microtiter plates, in the presence of culture medium, which is a saline solution with 0.85% sodium chloride, and a dehydrogenase activity indicator reagent, which is 2,3,5-triphenyltetrazolium chloride (denoted TTC). The results are presented in the following pages with the variation of the microbial population over the 28 days of study, for the tested microbial strain.

The measurements of microbial populations in the samples collected at each time, are carried out according to the following microtiter method, for one sample collection. In the 96 wells with a volume of 250 μL of a microtiter plate, 20 μL of the collected sample are diluted by a factor of 10 by dispersion in 180 μL of Letheen broth (Difco, ref. 268110) containing 1.5% of Tween 80 (Sigma, ref. P1754) and TTC (Sigma, ref. T8877). The microplate is incubated for 48 hours at 32.5° C., and the growth of the microorganisms is monitored by the color change, from colorless to red/pink. The highest reciprocal dilution indicating a growth makes it possible to determine the log number of each microorganism at each time.

The measurements of microbial populations taken at each collection time are expressed as colony forming units/ml.

TABLE 1
Bactericidal effect of isosorbide on the Propionobacterium acnes strain

| | | Number of colony forming strains after: | | | | |
|---|---|---|---|---|---|---|
| Dose | Inoculum | 24 h | 7 days | 14 days | 21 days | 28 days |
| Positive | 3.98 × 10⁵ | 4.00 × 10³ | 7.00 × 10⁴ | 4.00 × 10⁴ | 1.00 × 10⁴ | 1.00 × 10⁴ |
| control | | | | | | |
| Negative | 3.98 × 10⁵ | 0 | 0 | 0 | 0 | 0 |
| control | | | | | | |
| 1% | 3.98 × 10⁵ | 0 | 0 | 0 | 0 | 0 |
| 5% | 3.98 × 10⁵ | 0 | 0 | 0 | 0 | 0 |
| 9% | 3.98 × 10⁵ | 0 | 0 | 0 | 0 | 0 |

After 24 hours, no colony forming strain is observed: isosorbide made it possible to kill the entire inoculum. Isosorbide is a bactericide of the Propionobacterium acnes strain.

### Example 2: In Vitro Measurements of the Effect of Isosorbide on Microbial Strains that Cause Dandruff

The samples are prepared under sterile conditions and deposited in microplates (2-ml wells) with the concentrations hereunder. Two analysis controls were produced:

a positive control corresponding to 0.5% Phenonip®;

a negative control consisting of 0.85% physiological saline solution.

Samples and controls are contaminated by a Malassezia furfur fungal strain with around 1.50×10⁴ to 3.80×10⁴ colony forming units per milliliter, denoted cfu/ml. After contamination, the samples are carefully mixed by suction-discharge cycles in order to ensure a uniform distribution of the microorganism. The whole is incubated at 22° C. during 28 days.

The microbial population is sampled and counted at 24 hours, 7, 14, 21 and 28 days for the bacterial strain. The contaminated samples are collected and then deposited in serial dilutions in microtiter plates, in the presence of culture medium, which is a saline solution with 0.85% sodium chloride, and a dehydrogenase activity indicator reagent, which is 2,3,5-triphenyltetrazolium chloride (denoted TTC). The results are presented in the following pages with the variation of the microbial population over the 28 days of study, for the tested microbial strain.

The measurements of microbial populations in the samples collected at each time, are carried out according to the following microtiter method, for one sample collection. In the 96 wells with a volume of 250 μL of a microtiter plate, 20 μL of the collected sample are diluted by a factor of 10 by dispersion in 180 μL of Letheen broth (Difco, ref. 268110) containing 1.5% of Tween 80 (Sigma, ref. P1754) and TTC (Sigma, ref. T8877). The microplate is incubated for 48 hours at 32.5° C., and the growth of the microorganisms is monitored by the color change, from colorless to red/pink. The highest reciprocal dilution indicating a growth makes it possible to determine the log number of each microorganism at each time.

The measurements of microbial populations taken at each collection time are expressed as colony forming units/ml.

TABLE 2
Antifungal effect of isosorbide on the Malassezia furfur strain

| Dose of | | Number of colony forming strains after: | | | | |
|---|---|---|---|---|---|---|
| isosorbide | Innoculum | 24 h | 7 days | 14 days | 21 days | 28 days |
| Positive | 1.5 × 10⁴ | 4.00 × 10³ | 1.00 × 10³ | 7.00 × 10² | 1.00 × 10³ | 1.00 × 10³ |
| control | | | | | | |
| Negative | 1.5 × 10⁴ | 0 | 0 | 0 | 0 | 0 |
| control | | | | | | |
| 1% | 1.5 × 10⁴ | 0 | 0 | 0 | 0 | 0 |
| 5% | 1.5 × 10⁴ | 0 | 0 | 0 | 0 | 0 |
| 9% | 1.5 × 10⁴ | 0 | 0 | 0 | 0 | 0 |

After 24 hours, no colony forming strain is observed: isosorbide made it possible to kill the entire inoculum. Isosorbide is a bactericide of the Malassezia furfur strain.

### Example 3: In Vitro Measurements of the Effect of Isosorbide on Microbial Strains that Cause Bad Body Odors

The samples are prepared under sterile conditions and deposited in microplates (2-ml wells) with the concentrations hereunder. Two analysis controls were produced:

a positive control corresponding to 0.5% Phenonip®;

a negative control consisting of 0.85% physiological saline solution.

Samples and controls are contaminated by three bacterial strains, Staphylococcus epidermidis, Corynebacterium xerosis and Propionobacterium acnes, with around 1.43×10⁵ to 4.15×10⁵ colony forming units per milliliter, denoted cfu/ml. After contamination, the samples are carefully mixed by suction-discharge cycles in order to ensure a uniform distribution of the microorganism. The whole is incubated at 22° C. during 28 days.

The microbial population is sampled and counted at 24 hours, 7, 14, 21 and 28 days for each bacterial strain. The contaminated samples are collected and then deposited in serial dilutions in microtiter plates, in the presence of culture medium, which is a saline solution with 0.85% sodium chloride, and a dehydrogenase activity indicator reagent, which is 2,3,5-triphenyltetrazolium chloride (denoted TTC). The results are presented in the following pages with the variation of the microbial population over the 28 days of study, for the tested microbial strain.

The measurements of microbial populations in the samples collected at each time, are carried out according to the following microtiter method, for one sample collection. In the 96 wells with a volume of 250 μL of a microtiter plate, 20 μL of the collected sample are diluted by a factor of 10 by dispersion in 180 μL of Letheen broth (Difco, ref. 268110) containing 1.5% of Tween 80 (Sigma, ref. P1754) and TTC (Sigma, ref. T8877). The microplate is incubated for 48 hours at 32.5° C., and the growth of the microorganisms is monitored by the color change, from colorless to red/pink. The highest reciprocal dilution indicating a growth makes it possible to determine the log number of each microorganism at each time.

The measurements of microbial populations taken at each collection time are expressed as colony forming units/ml.

TABLE 3
Bactericidal effect of isosorbide on the Corynobacterium xerosis strain

| | | Number of colony forming strains after: | | | | |
|---|---|---|---|---|---|---|
| Dose | Inoculum | 24 h | 7 days | 14 days | 21 days | 28 days |
| Positive | 3.2 × 10⁵ | 7.00 × 10⁴ | 1.00 × 10⁴ | 7.00 × 10³ | 4.00 × 10³ | 7.00 × 10³ |
| control | | | | | | |
| Negative | 3.2 × 10⁵ | 0 | 0 | 0 | 0 | 0 |
| control | | | | | | |
| 1% | 3.2 × 10⁵ | 4 × 10² | 0 | 0 | 0 | 0 |
| 5% | 3.2 × 10⁵ | 1 × 10² | 0 | 0 | 0 | 0 |
| 9% | 3.2 × 10⁵ | 3.7 × 10² | 0 | 0 | 0 | 0 |

After 24 hours, the number of colony forming strains is reduced by at least 3 log 10 for the three doses tested. After 7 days, there is not any more colony forming strain: isosorbide is a moderate bactericide of the Corynobacterium xerosis strain.

TABLE 4
Bactericidal effect of isosorbide on the Staphylococcus epidermidis strain

| | | Number of colony forming strains after: | | | | |
|---|---|---|---|---|---|---|
| Dose | Inoculum | 24 h | 7 days | 14 days | 21 days | 28 days |
| Positive | 1.43 × 10⁵ | 7.00 × 10⁴ | 4.00 × 10⁴ | 7.00 × 10⁴ | 1.00 × 10⁵ | 1.00 × 10⁵ |
| control | | | | | | |
| Negative | 1.43 × 10⁵ | 0 | 0 | 0 | 0 | 0 |
| control | | | | | | |
| 1% | 1.43 × 10⁵ | 4 × 10³ | 0 | 0 | 0 | 0 |
| 5% | 1.43 × 10⁵ | 1 × 10³ | 0 | 0 | 0 | 0 |
| 9% | 1.43 × 10⁵ | 7 × 10² | 0 | 0 | 0 | 0 |

After 24 hours, the number of colony forming strains is reduced by at least 2 log 10 for the three doses tested. After 7 days, there is not any colony forming strain: isosorbide is a weak bactericide of the Staphylococcus epidermidis strain.

TABLE 5
Bactericidal effect of isosorbide on the Propionobacterium acnes strain

| | | Number of colony forming strains after: | | | | |
|---|---|---|---|---|---|---|
| Dose | Inoculum | 24 h | 7 days | 14 days | 21 days | 28 days |
| Positive | 3.98 × 10⁵ | 4.00 × 10³ | 7.00 × 10⁴ | 4.00 × 10⁴ | 1.00 × 10⁴ | 1.00 × 10⁴ |
| control | | | | | | |
| Negative | 3.98 × 10⁵ | 0 | 0 | 0 | 0 | 0 |
| control | | | | | | |
| 1% | 3.98 × 10⁵ | 0 | 0 | 0 | 0 | 0 |
| 5% | 3.98 × 10⁵ | 0 | 0 | 0 | 0 | 0 |
| 9% | 3.98 × 10⁵ | 0 | 0 | 0 | 0 | 0 |

After 24 hours, no colony forming strain is observed: isosorbide made it possible to kill the entire inoculum. Isosorbide is a bactericide of the Propionobacterium acnes strain.

The invention is not limited to the examples described above, given only by way of example, but it encompasses all the alternatives that a person skilled in the art could contemplate in the context of the sought protection.

1. A use of at least one dianhydrohexitol as bacteriostatic and/or bactericidal or/and antifungal agent.

2. The use of at least one dianhydrohexitol according to claim 1, wherein it has:
bacteriostatic and/or bactericidal action on a bacterial strain selected among the Propionobacterium spp family, preferentially on the Propionobacterium acnes bacterial strain and/or on a strain of the Corynebacterium spp family, preferentially on the Corynebacterium xerosis bacterial strain, and/or on a strain of the Staphylococcus spp family, preferentially on the Staphylococcus epidermidis bacterial strain, and/or
action on a strain of the Malassezia spp family, preferentially on the Malassezia furfur strain.

3. The use of at least one dianhydrohexitol according to claim 1, wherein the dianhydrohexitol is selected among isosorbide, isomannide, isoidide, preferentially isosorbide.

4. The use of at least one dianhydrohexitol according to claim 1, wherein the dianhydrohexitol concentration applied to the skin is at least 1 mg/cm², preferentially at least 25 mg/cm², and most preferentially at least 50 mg/cm².

5. A non-therapeutic cosmetic use of at least one dianhydrohexitol according to claim 1, to promote the elimination of the cosmetic effects of acne.

6. The non-therapeutic cosmetic use according to claim 5, wherein the cosmetic effects of acne are selected among inflammation, or itching.

7. A dianhydrohexitol, for therapeutic use thereof in the treatment of acne.

8. The non-therapeutic cosmetic use according to claim 1, of at least one dianhydrohexitol to promote the elimination of the cosmetic effects of excessive flaking.

9. The non-therapeutic cosmetic use according to claim 8, wherein the cosmetic effects of excessive flaking are selected among inflammation, or itching.

10. A dianhydrohexitol, for therapeutic use thereof in the treatment of dandruff.

11. The non-therapeutic cosmetic use of at least one dianhydrohexitol according to claim 1, to reduce or prevent the formation of bad body odors, preferentially the bad odors resulting from the breakdown of sweat.

12. A dianhydrohexitol, for therapeutic use thereof in the treatment of bad body odors.

13. A non-therapeutic cosmetic method for caring for the skin or the scalp, comprising the following steps:
cleaning the skin or the scalp,
applying to the skin or scalp a cosmetic or dermatological preparation comprising at least one dianhydrohexitol, preferentially isosorbide, before, during or after the appearance of the cosmetic effects of acne or bad odors or dandruff, for a duration of at least 20 minutes, preferentially at least 2 hours, and most preferentially at least 6 hours,
removing said preparation.

14. The non-therapeutic cosmetic method according to claim 13, for skin that is susceptible to bad odors, in particular linked to the breakdown of sweat by the cutaneous microbiome, wherein said preparation is applied to the skin under the arms or to the groin.
